(19) United States
(12) Patent Application Publication
(10) Pub. No.: US 2021/0396765 A1
(43) Pub. Date: Dec. 23, 2021

(54) DETECTION AND TREATMENT OF IL-17 AND IL-13 RELATED CONDITIONS

(71) Applicant: The Cleveland Clinic Foundation, Cleveland, OH (US)

(72) Inventors: Xiaoxia Li, Solon, OH (US); Katarzyna Bulek, Cleveland Heights, OH (US); Caini Liu, Solon, OH (US); Mark Aronica, Chagrin Falls, OH (US); Liz Hong, Cleveland Heights, OH (US)

(21) Appl. No.: 17/152,331

(22) Filed: Jan. 19, 2021

Related U.S. Application Data
(63) Continuation of application No. 16/073,522, filed on Jul. 27, 2018, now abandoned.
(60) Provisional application No. 62/290,535, filed on Feb. 3, 2016.

(51) Int. Cl. G01N 33/68; G01N 33/53

(52) U.S. Cl. CPC G01N 33/6869; G01N 33/53; G01N 33/68; G01N 2800/24; G01N 2800/122; G01N 2333/54; G01N 2333/5437; G01N 2800/52

(57) ABSTRACT

Provided herein are compositions, systems, and methods for detecting and treating IL-17 and/or IL-13 related conditions, such as asthma, autoimmune diseases, and cancer. In some embodiments, methods, compositions, and systems are provided for detecting elevated protein or RNA levels of IL-17 (and/or IL-13) and a second marker selected from: SAA, LCN2, and YKL-40, and treating the subject with an IL-17 (or IL-13) inhibitor or IL-17 (or IL-13) receptor inhibitor. In other embodiments, methods, compositions, and systems are provided for detecting a biomarker in a sample from a subject that has been treated with an IL-17 (or IL-13) inhibitor or IL-17 (or IL-13) receptor inhibitor, where the biomarker is SAA, LCN2, or YKL-40, and where at least one action is performed, such as identifying elevated levels of the biomarker in the biological sample and treating the subject with an IL-17 (or IL-13) inhibitor and/or IL-17 (or IL-13) receptor inhibitor.

### CROSS-REFERENCE TO RELATED APPLICATIONS

The present application is a continuation of U.S. patent application Ser. No. 16/073,522, filed Jul. 27, 2018, which is a § 371 U.S. National Entry Application of PCT/US2017/016127, which claims the priority benefit of U.S. Provisional Patent Application 62/290,535, filed Feb. 3, 2016, each of which is incorporated by reference in its entirety.

### STATEMENT REGARDING FEDERAL FUNDING

This invention was made with government support under HL103453 awarded by the National Institutes of Health. The government has certain rights in the invention.

### FIELD

Provided herein are compositions, systems, and methods for detecting and treating IL-17 and/or IL-13 related conditions, such as asthma, autoimmune diseases, and cancer. In some embodiments, methods, compositions, and systems are provided for detecting elevated protein or RNA levels of IL-17 (and/or IL-13) and a second marker selected from: SAA, LCN2, and YKL-40, and treating the subject with an IL-17 (or IL-13) inhibitor or IL-17 (or IL-13) receptor inhibitor. In other embodiments, methods, compositions, and systems are provided for detecting a biomarker in a sample from a subject that has been treated with an IL-17 (or IL-13) inhibitor or IL-17 (or IL-13) receptor inhibitor, where the biomarker is SAA, LCN2, or YKL-40, and where at least one action is performed, such as identifying elevated levels of the biomarker in the biological sample and treating the subject with an IL-17 (or IL-13) inhibitor and/or IL-17 (or IL-13) receptor inhibitor.

### BACKGROUND

Approximately 38.4 million Americans have been diagnosed with asthma by a health professional during their lifetime. This chronic inflammatory disease places a significant burden on both the health care system and individual patients, with annual expenditures for health and lost productivity due to asthma estimated at over $20 billion. In spite of high morbidity and costs, most asthmatic patients have mild-to-moderate disease and about 5-8% of asthmatic patients fall into the category of “chronic severe asthma” (CSA). Asthmatic patients have significant reduction in quality of life as a result of their asthma, have frequent hospital admissions and emergency visits, and account for a much larger percentage of overall health care costs. Clinically, asthma is characterized by a component of irreversible airflow obstruction and peripheral airways disease, ongoing mediator release and a reduced association with atopy. There is currently no way to predict whether an individual patient with asthma will be stable over time, or exhibit declining lung function that leads to development of CSA.

Current asthma treatment and diagnosis are predominantly clinically based, or use pulmonary function testing, which is expensive, can only occur in specialized pulmonary function testing laboratories, and is inconvenient. Exhaled breath NO is another recent test used in asthma, but it lacks specificity and sensitivity for diagnosis and monitoring of asthma treatment/severity. Further, it is not readily collected and sent to laboratory for testing, like most other diagnostic tests. Rather, it takes specialized instrumentation to be located on-site for testing. Simple reliable and objective quantitative measures of blood or urine based tests for the diagnosis of asthma, and for predicting risk of exacerbation, need for therapeutic titration, or monitoring of response to therapeutic interventions, are needed.

### SUMMARY

Provided herein are compositions, systems, and methods for detecting and treating IL-17 and/or IL-13 related conditions, such as asthma, autoimmune diseases, and cancer. In some embodiments, methods, compositions, and systems are provided for detecting elevated protein or RNA levels of IL-17 (and/or IL-13) and a second marker selected from: SAA, LCN2, and YKL-40, and treating the subject with an IL-17 (or IL-13) inhibitor or IL-17 (or IL-13) receptor inhibitor. In other embodiments, methods, compositions, and systems are provided for detecting a biomarker in a sample from a subject that has been treated with an IL-17 (or IL-13) inhibitor or IL-17 (or IL-13) receptor inhibitor, where the biomarker is SAA, LCN2, or YKL-40, and where at least one action is performed, such as identifying elevated levels of the biomarker in the biological sample and treating the subject with an IL-17 (or IL-13) inhibitor and/or IL-17 (or IL-13) receptor inhibitor.

In some embodiments, provided herein are methods comprising: a) detecting first and second biomarker levels (e.g., protein and/or mRNA levels) in a biological sample (e.g., blood sample, plasma, serum, urine, etc.) from a subject, wherein the first biomarker is IL-17, wherein the second biomarker is selected from the group consisting of human SAA, LCN2, YKL-40, IL-13, Csf3, Plat, Lgals3, Prl2c2, S100a8, and Gdf6 (and/or a gene listed in Table 1), and wherein the subject has symptoms of an inflammatory condition, autoimmune disease, or cancer, b) identifying elevated levels of both the first and second biomarkers in the biological sample (e.g., by comparison to a control value); and c) treating the subject with an IL-17 inhibitor (e.g., COSENTYX (secukinumab), Ixekizumab, or MSB0010841), and/or an IL-17 receptor inhibitor (e.g., brodalumab), such that the subject's symptoms of the inflammatory condition, autoimmune disease, or cancer are reduced or eliminated. In certain embodiments, the particular biomarker levels are determined to be elevated by comparison to a control known to not have elevated levels of that particular biomarker (e.g., control value set based on the general population, or a select population of apparently non-diseased subjects).

In certain embodiments, provided herein are methods comprising: a) detecting a biomarker level (e.g., protein or mRNA) in a biological sample (e.g., blood, serum, plasma, urine, etc.) from a subject that has been treated with an exogenous IL-17 inhibitor and/or exogenous IL-17 receptor inhibitor, wherein the biological sample comprises the exogenous IL-17 inhibitor and/or the exogenous IL-17 receptor inhibitor, wherein the biomarker is selected from the group consisting of human SAA, LCN2, YKL-40, IL-13, Csf3, Plat, Lgals3, Prl2c2, S100a8, and Gdf6, and b) performing at least one of the following: i) identifying elevated levels of the biomarker in the biological sample (e.g., by comparison to a control value), and treating the subject with an IL-17 inhibitor (e.g., COSENTYX (secukinumab), Ixekizumab, or MSB0010841) and/or an IL-17 receptor inhibitor (e.g., brodalumab); ii) identifying elevated levels of the biomarker in the biological sample, and prescribing the subject (e.g., a human subject) an IL-17 inhibitor and/or an IL-17 receptor inhibitor; iii) generating and/or transmitting a report that indicates the biomarker is elevated in the subject and that the subject is in need of an IL-17 inhibitor and/or IL-17 receptor inhibitor; iv) identifying non-elevated levels of the biomarker in the biological sample, and discontinuing further treatment with the exogenous IL-17 inhibitor and/or the exogenous IL-17 receptor inhibitor; and v) generating and/or transmitting a report that indicates the biomarker is non-elevated in the subject and that the subject is not in need of any further IL-17 inhibitor and/or IL-17 receptor inhibitor. In certain embodiments, the inflammatory condition is selected from asthma (e.g., mild asthma, severe asthma, etc.), cancer, or an autoimmune disease, or other inflammatory condition.

In some embodiments, provided herein are methods comprising: a) selecting a subject with symptoms of an inflammatory condition, that also has elevated levels of both IL-17 and a marker selected from the group consisting of human SAA, LCN2, YKL-40, IL-13, Csf3, Plat, Lgals3, Prl2c2, S100a8, and Gdf6 or marker from Table 1, or combinations thereof; and b) treating the subject with an IL-17 inhibitor, and/or an IL-17 receptor inhibitor, such that the subject's symptoms of the inflammatory condition are reduced or eliminated.

In further embodiments, provided herein are methods comprising: a) selecting a subject that: i) has been treated with an IL-17 inhibitor and/or IL-17 receptor inhibitor, and ii) has an elevated level of a biomarker selected from the group consisting of: human SAA, LCN2, YKL-40, IL-13, Csf3, Plat, Lgals3, Prl2c2, S100a8, and Gdf6; and b) treating the subject (e.g., human subject) with an IL-17 inhibitor, and/or an IL-17 receptor inhibitor. In certain embodiments, the inflammatory condition is selected from asthma (mild asthma or severe asthma), cancer, or an autoimmune disease, or other inflammatory disease.

In particular embodiments, provided herein are systems comprising: a) a composition comprising an IL-17 inhibitor, and/or an IL-17 receptor inhibitor, and b) a report (e.g., printed or electronic report) that indicates that a subject with symptoms of an inflammatory condition has an elevated level of IL-17 and/or an elevated level of a biomarker selected from the group consisting of: human SAA, LCN2, YKL-40, IL-13, Csf3, Plat, Lgals3, Prl2c2, S100a8, and Gdf6, or a marker from Table 1 or combinations thereof.

In further embodiments, provided herein are system comprising: a) a composition comprising an IL-17 inhibitor, and/or an IL-17 receptor inhibitor, and b) a report (e.g., printed or electronic) that indicates that a biological sample from a subject has an elevated level of a biomarker (e.g., protein level or mRNA level) selected from the group consisting of: SAA, LCN2, YKL-40, IL-13, Csf3, Plat, Lgals3, Prl2c2, S100a8, and Gdf6, or a marker from Table 1 or combinations thereof. In certain embodiments, the inflammatory condition is selected from asthma (mild asthma or severe asthma), cancer, or an autoimmune disease, or other inflammatory disease.

In some embodiments, provided herein are systems comprising: a) components of a first assay (e.g., antibodies particular for a particular marker protein, or nucleic acid probes specific for a particular marker mRNA sequence, and associated components such as buffer, detectable moieties, etc.) for detecting IL-17 in a biological sample from a subject; b) components of a second assay for detecting a marker in a biological sample from the subject selected from the group consisting of: human SAA, LCN2, YKL-40, IL-13, Csf3, Plat, Lgals3, Prl2c2, S100a8, and Gdf6 or a marker from Table 1 or combinations thereof and c)

at least one of the following: i) a composition comprising an IL-17 inhibitor and/or an IL-17 receptor inhibitor, and ii) a biological sample from the subject, wherein the biological sample comprises an exogenous IL-17 inhibitor and/or an exogenous IL-17 receptor inhibitor.

In certain embodiments, provided herein are systems comprising: a) components of an assay for detecting a marker in a biological sample from a subject selected from the group consisting of: human SAA, LCN2, YKL-40, IL-13, Csf3, Plat, Lgals3, Prl2c2, S100a8, and Gdf6 or a marker from Table 1 or combinations thereof; and b) at least one of the following: i) a composition comprising an IL-17 inhibitor and/or an IL-17 receptor inhibitor, and ii) a biological sample from the subject (e.g., blood, plasma, serum, urine, etc.), wherein the biological sample comprises an exogenous IL-17 inhibitor and/or an exogenous IL-17 receptor inhibitor. In some embodiments, the subject (e.g., human subject) had symptoms of an inflammatory condition prior to being treated with the IL-17 inhibitor and/or the IL-17 receptor inhibitor. In certain embodiments, the particular biomarker levels are determined to be elevated by comparison to a control known to not have elevated levels of that particular biomarker (e.g., control value set based on the general population, or a select population of apparently non-diseased subjects).

In some embodiments, provided herein are methods comprising: a) detecting first and second biomarker levels (e.g., protein or mRNA levels) in a biological sample (e.g., whole blood, serum, plasma, urine, etc.) from a subject, wherein the first biomarker is IL-13, wherein the second biomarker is selected from the group consisting of human SAA, LCN2, YKL-40, IL-17, Csf3, Plat, Lgals3, Prl2c2, S100a8, and Gdf6 or a marker from Table 1 or combinations thereof, and wherein the subject has symptoms of an inflammatory condition, b) identifying elevated levels of both the first and second biomarkers in the biological sample (e.g., by comparison to a control value); and c) treating the subject with an IL-13 inhibitor (e.g., Lebrikizumab IL-13 targeting antibody or other IL-13 antibody), and/or an IL-13 receptor inhibitor, such that the subject's symptoms of the inflammatory condition are reduced or eliminated. In certain embodiments, the particular biomarker levels are determined to be elevated by comparison to a control known to not have elevated levels of that particular biomarker (e.g., control value set based on the general population, or a select population of apparently non-diseased subjects).

In particular embodiments, provided herein are methods comprising: a) detecting a biomarker level (e.g., protein or mRNA levels) in a biological sample from a subject that has been treated with an exogenous IL-13 inhibitor (e.g., Lebrikizumab IL-13 targeting antibody or other IL-13 antibody) and/or exogenous IL-13 receptor inhibitor (e.g., monoclonal antibody GSK679586 or similar antibody), wherein the biological sample comprises the exogenous IL-13 inhibitor and/or the exogenous IL-13 receptor inhibitor, wherein the biomarker is selected from the group consisting of human SAA, LCN2, YKL-40, IL-17, Csf3, Plat, Lgals3, Prl2c2, S100a8, and Gdf6 or a marker from Table 1 or combinations thereof, and b) performing at least one of the following: i) identifying elevated levels of the biomarker in the biological sample (e.g., via comparison to a control), and treating the subject with an IL-13 inhibitor and/or an IL-13 receptor inhibitor; ii) identifying elevated levels of the biomarker in the biological sample, and prescribing the subject an IL-13 inhibitor (e.g., Lebrikizumab IL-13 targeting antibody or other IL-13 antibody) and/or an IL-13 receptor inhibitor (e.g., monoclonal antibody GSK679586 or similar antibody); iii) generating and/or transmitting a report (e.g., printed or electronic, such as in an email) that indicates the biomarker is elevated in the subject and that the subject is in need of an IL-13 inhibitor (e.g., Lebrikizumab IL-13 targeting antibody or other IL-13 antibody) and/or IL-13 receptor inhibitor (e.g., monoclonal antibody GSK679586 or similar antibody); iv) identifying non-elevated levels (e.g., in comparison to a control) of the biomarker in the biological sample, and discontinuing further treatment with the exogenous IL-13 inhibitor and/or the exogenous IL-13 receptor inhibitor; and v) generating and/or transmitting a report that indicates the biomarker is non-elevated in the subject and that the subject is not in need of any further IL-13 inhibitor and/or IL-13 receptor inhibitor.

In other embodiments, provided herein are methods comprising: a) selecting a subject with symptoms of an inflammatory condition, that also has elevated levels of both IL-13 and a marker selected from the group consisting of human SAA, LCN2, YKL-40, IL-17, Csf3, Plat, Lgals3, Prl2c2, S100a8, and Gdf6 or a marker in Table 1 or combinations thereof; and b) treating the subject with an IL-13 inhibitor (e.g., Lebrikizumab IL-13 targeting antibody or other IL-13 antibody), and/or an IL-13 receptor inhibitor (e.g., monoclonal antibody GSK679586 or similar antibody), such that the subject's symptoms of the inflammatory condition are reduced or eliminated.

In some embodiments, provided herein are methods comprising: a) selecting a subject that: i) has been treated with an exogenous IL-13 inhibitor (e.g., Lebrikizumab IL-13 targeting antibody or other IL-13 antibody) and/or exogenous IL-13 receptor inhibitor (e.g., monoclonal antibody GSK679586 or similar antibody), and ii) has an elevated level of a biomarker selected from the group consisting of: human SAA, LCN2, YKL-40, IL-17, Csf3, Plat, Lgals3, Prl2c2, S100a8, and Gdf6 or a marker from Table 1 or combinations there; and b) treating the subject with an IL-13 inhibitor (e.g., Lebrikizumab IL-13 targeting antibody or other IL-13 antibody), and/or an IL-13 receptor inhibitor (e.g., monoclonal antibody GSK679586 or similar antibody).

In certain embodiments, provided herein are systems comprising: a) a composition comprising an IL-13 inhibitor (e.g., Lebrikizumab IL-13 targeting antibody or other IL-13 antibody), and/or an IL-13 receptor inhibitor (e.g., monoclonal antibody GSK679586 or similar antibody), and b) a report that indicates that a subject with symptoms of an inflammatory condition has an elevated level of IL-13 and an elevated level of biomarker selected from the group consisting of: human SAA, LCN2, YKL-40, IL-17, Csf3, Plat, Lgals3, Prl2c2, S100a8, and Gdf6 or a marker from Table 1 or combinations thereof.

In some embodiments, provided herein are systems comprising: a) a composition comprising an IL-13 inhibitor (e.g., Lebrikizumab IL-13 targeting antibody or other IL-13 antibody), and/or an IL-13 receptor inhibitor (e.g., monoclonal antibody GSK679586 or similar antibody), and b) a report that indicates that a biological sample from a subject has an elevated level of a biomarker selected from the group consisting of: human SAA, LCN2, YKL-40, IL-17, Csf3, Plat, Lgals3, Prl2c2, S100a8, and Gdf6 or a marker from Table 1 or combinations thereof.

In other embodiments, provided herein are systems comprising: a) components of a first assay for detecting IL-13 (e.g., nucleic acid probe hybridizing to IL-13 mRNA or antibody that binds IL-13 protein, buffer, signal component, etc.) in a biological sample from a subject; b) components of a second assay for detecting a marker (e.g., nucleic acid probe hybridizing to the recited marker mRNA or antibody that binds the recited marker protein, buffer, signal component, etc.) in a biological sample from the subject selected from the group consisting of: human SAA, LCN2, YKL-40, IL-17, Csf3, Plat, Lgals3, Prl2c2, S100a8, and Gdf6 or a marker from Table 1 or combination thereof and c) at least one of the following: i) a composition comprising an IL-13 inhibitor (e.g., Lebrikizumab IL-13 targeting antibody or other IL-13 antibody) and/or an IL-13 receptor inhibitor (e.g., monoclonal antibody GSK679586 or similar antibody), and ii) a biological sample from the subject, wherein the biological sample comprises an exogenous IL-13 inhibitor and/or an exogenous IL-13 receptor inhibitor.

In certain embodiments, provided herein are systems comprising: a) components of an assay for detecting a marker in a biological sample from a subject selected from the group consisting of: human SAA, LCN2, YKL-40, IL-17, Csf3, Plat, Lgals3, Prl2c2, S100a8, and Gdf6 or a marker from Table 1 or combinations thereof and b) at least one of the following: i) a composition comprising an IL-13 inhibitor (e.g., Lebrikizumab IL-13 targeting antibody or other IL-13 antibody) and/or an IL-13 receptor inhibitor (e.g., monoclonal antibody GSK679586 or similar antibody), and ii) a biological sample from the subject, wherein the biological sample comprises an exogenous IL-13 inhibitor and/or an exogenous IL-13 receptor inhibitor.

In some embodiments, the biomarkers herein (e.g., SAA, LCN2, YKL-40, IL-17, IL-13, Csf3, Plat, Lgals3, Prl2c2, S100a8, and Gdf6 or a marker from Table 1 or combinations thereof) are detected and/or quantitated in a biological sample obtained from a subject. A subject may be a human, non-human primate, mouse, rat, or other mammalian subject. In some embodiments, a biological sample comprises urine or a urine product, blood or a blood product (e.g., serum, plasma, or whole blood, etc.), tears, or other body fluids or tissues (e.g., from a human subject). A sample may be processed (e.g., concentrated, diluted, salt-depleted, precipitated, lysed, extracted, centrifuged, denatured, etc.) or unprocessed.

In embodiments in which a biomarker (e.g., SAA, LCN2, YKL-40, IL-17, Csf3, Plat, Lgals3, Prl2c2, S100a8, and Gdf6 or a marker from Table 1 or combinations thereof) is a protein, polypeptide and/or peptide, detection and/or quantification reagents may comprise antibodies or antibody-like reagents, aptamers, etc. that bind (e.g., specifically) to the specific biomarker. In such embodiments, detection and/or quantification may be achieved by, for example, an immunoassay, Western blot, enzyme-linked immunosorbent assay (ELISA), radioimmunoassay (RIA), fluorimetric assay, or other suitable assays known in the field.

In some embodiments, the biomarkers (e.g., SAA, LCN2, YKL-40, IL-17, Csf3, Plat, Lgals3, Prl2c2, S100a8, and Gdf6 or a marker from Table 1 or combinations thereof) are RNAs (e.g., mRNA) encoding proteins or subunits thereof. In embodiments in which a biomarker is an RNA (e.g., mRNA), detection and/or quantification reagents may comprise primers (e.g., for amplification, reverse transcription, etc.) or probes (e.g., detectably-labeled (e.g., optically-labeled, fluorescently labeled, etc.) oligonucleotides) that bind (e.g., specifically) to the biomarker. In such embodiments, detection and/or quantification may be achieved by, for example, RT-PCR, qPCR, Northern blot analysis, an enzymatic cleavage assay (e.g., INVADER, Hologic, Inc.; See e.g., U.S. Pat. Nos. 5,846,717, 6,090,543; 6,001,567; 5,985,557; and 5,994,069; each of which is herein incorporated by reference), a hybridization assay (e.g., TaqMan assay (Life Technologies; See e.g., U.S. Pat. Nos. 5,962,233 and 5,538,848, each of which is herein incorporated by reference), etc.

In some embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some embodiments, quantitative PCR (qPCR) or real time PCR (RT-PCR) is used to detect/quantify biomarkers. In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR produces an absolute measurement such as number of copies of mRNA in a sample or portion of a sample.

In some embodiments, nucleic acid from a sample is sequenced (e.g., in order to detect biomarkers). Nucleic acid molecules may be sequence analyzed by any number of techniques. The analysis may identify the sequence of all or a part of a nucleic acid. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack, experimentally RNA is usually, although not necessarily, reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties) and the Adessi PCR colony technology (Adessi et al. (2000). Nucleic Acid Res. 28, E87; WO 00018957; herein incorporated by reference in its entirety).

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, Pacific Biosciences (PAC BIO RS II) and other platforms commercialized.

In some embodiments, a detection and/or capture reagent is an oligonucleotide probe comprising a portion that is complementary to encoding a biomarker protein (e.g., SAA, LCN2, YKL-40, IL-17, Csf3, Plat, Lgals3, Prl2c2, S100a8, and Gdf6 or a marker from Table 1). For example, provided herein are nucleic acid oligonucleotides comprising a portion with at least 70% sequence identity (e.g., 70%, 75%, 80%, 85%, 90%, 91%, 92%, 93%, 94%, 95%, 96%, 97%, 98%, 99%, 100%, or ranges therein) with one of SAA, LCN2, YKL-40, IL-17, Csf3, Plat, Lgals3, Prl2c2, S100a8, and Gdf6 or a marker from Table 1 (e.g., 8 nt, 10 nt, 12 nt, 15 nt, 18 nt, 20 nt, 25 nt, 30 nt, 35 nt, 40 nt, 50 nt, 75 nt, 100 nt, or more, or ranges therein. In some embodiments, oligonucleotides are primers for amplifying a portion of a biomarker target RNA or DNA sequence. In some embodiments, oligonucleotides are probes (e.g., detectably labeled (e.g., fluorescently labeled), etc.) for detecting/quantifying all or a portion of a target biomarker RNA or DNA sequence.

In some embodiments, provided herein are methods for detecting a protein biomarker in a subject sample by exposing biological sample from a human subject (e.g., urine, blood, plasma, or serum) to detection reagents (e.g., antibodies, aptamers, etc.) for one or more of SAA, LCN2, YKL-40, IL-17, Csf3, Plat, Lgals3, Prl2c2, S100a8, and Gdf6 or a marker from Table 1, with subsequent semiquantification or quantification using a suitable assay not limited to immunoblotting, enzyme-linked immunosorbent assay, or fluorescent immunoassay. In some embodiments, methods further comprise immunoblotting, enzyme-linked immunosorbent assay, or fluorescent immunoassay of said sample of a human subject with said detection reagents (e.g., antibodies, aptamers, etc.) for one or more of SAA, LCN2, YKL-40, IL-17, Csf3, Plat, Lgals3, Prl2c2, S100a8, and Gdf6 or a marker from Table 1 or combinations thereof. In some embodiments, the sample (e.g., urine or blood from a human subject) is centrifuged at sub-ultracentrifugation speeds prior to exposure to said detection reagents. In some embodiments, the detection reagents are those used in the experiments conducted during development of embodiments described herein.

In some embodiments, detection of biomarkers (e.g., one or more of SAA, LCN2, YKL-40, IL-17, Csf3, Plat, Lgals3, Prl2c2, S100a8, and Gdf6 or a marker from Table 1) in a body fluid or tissue (e.g., blood, urine, etc.) is performed with one or more additional assays. In some embodiments, reagents for detecting biomarker (e.g., SAA, LCN2, YKL-40, IL-17, Csf3, Plat, Lgals3, Prl2c2, S100a8, and Gdf6 or a marker from Table 1) protein or mRNA is on a panel of biomarkers tested for determining responsiveness to treatment (e.g., treatment with IL-17 or IL-13 inhibitors or IL-17 or IL-13 receptor inhibitors). In some embodiments, provided herein are panels for detecting two or more markers (e.g., one or more of SAA, LCN2, YKL-40, IL-17, Csf3, Plat, Lgals3, Prl2c2, S100a8, and Gdf6 or a marker from Table 1).

In certain embodiments, provided herein are kits or systems comprising: a) components of a first assay for detecting IL-17 in a biological sample from a subject; b) components of a second assay for detecting a marker in a biological sample from said subject selected from the group consisting of: human SAA, LCN2, YKL-40, IL-13, Csf3, Plat, Lgals3, Prl2c2, S100a8, and Gdf6. In particular embodiments, the components of a first assay for detecting IL-17 comprises IL-17 monoclonal antibodies or antigen biding portions thereof. In other embodiments, the components of a the second assay for detecting a marker comprises monoclonal antibodies or antigen biding portions thereof selected from the group consisting of: anti-SAA, anti-LCN2, anti-YKL-40, anti-IL-13, anti-Csf3, anti-Plat, anti-Lgals3, anti-Prl2c2, anti-S100a8, and anti-Gdf6. In further embodiments, the kits and systems further comprise: c) at least one of the following: i) a composition comprising an IL-17 inhibitor and/or an IL-17 receptor inhibitor, and ii) a biological sample from said subject, wherein said biological sample comprises an exogenous IL-17 inhibitor and/or an exogenous IL-17 receptor inhibitor.

### BRIEF DESCRIPTION OF THE DRAWINGS

FIG. 1 shows real-time PCR analysis of Saa, Lcn2, C3 and Angptl4 in primary tracheal smooth muscle cells untreated or treated with IL-17A (50 ng/ml), IL-13 (long/ml) or IL-17A+IL-13 for 24 h.

FIG. 2A shows a statistical analysis performed using Mann-Whitney U test.

FIG. 2B shows a Pearson correlation test, on plasma samples collected from healthy controls (n=13), mild asthmatics (n=14) and severe asthmatics (n=14), where the productions of IL-17A, SAA, LCN2, YKL-40 and CRP were measured using ELISA.

FIG. 3 shows a heat map of significantly up-regulated secreted protein-coding genes 24 hours after IL-17A, IL-13 and IL-17A+13 stimulation in mouse ASM cells by RNA-Seq (P≤0.05, fold≥2).

FIG. 4 shows an exemplary patient report for a patient who is taking an IL-17 inhibitor.

FIG. 5 shows an exemplary patient report for a patient who is taking an IL-13 inhibitor.

### DEFINITIONS

As used herein, the terms “subject” refers to any animal (e.g., a mammal), including, but not limited to, humans, non-human primates, rodents, and the like.

As used herein, the term “reagent(s) capable of specifically detecting biomarker expression” refers to reagents used to detect the expression of biomarkers (e.g., SAA, LCN2, YKL-40, IL-17, IL-13, Csf3, Plat, Lgals3, Prl2c2, S100a8, and Gdf6 or a marker from Table 1 or combinations thereof. Examples of suitable reagents include but are not limited to, nucleic acid probes capable of specifically hybridizing to mRNA or cDNA, and antibodies (e.g., monoclonal antibodies).

As used herein, the term “sample” is used in its broadest sense. In one sense, it is meant to include a specimen or culture obtained from any source. Biological samples may be obtained from animals (including humans) and encompass fluids, solids, tissues, and gases. Biological samples include urine, saliva, tissues, lacrimal fluid, and blood products, such as plasma, serum and the like.

As used herein, the term “antibody” is used in the broadest sense and specifically covers human, non-human (e.g. murine) and humanized monoclonal antibodies (including full length monoclonal antibodies), polyclonal antibodies, multi-specific antibodies (e.g., bispecific antibodies), single-chain antibodies, and antibody fragments so long as they exhibit the desired biological activity.

As used herein, the term “probe” refers to an oligonucleotide (e.g., a sequence of nucleotides), whether occurring naturally as in a purified restriction digest or produced synthetically, recombinantly or by PCR amplification, that is capable of hybridizing to at least a portion of another oligonucleotide of interest (e.g., a biomarker). A probe may be single-stranded or double-stranded. Probes are useful in the detection, identification and isolation of particular gene sequences. It is contemplated that any probe used in the present invention may be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

### DETAILED DESCRIPTION

Provided herein are compositions, systems, and methods for detecting and treating IL-17 and/or IL-13 related conditions, such as asthma, autoimmune diseases, and cancer. In some embodiments, methods, compositions, and systems are provided for detecting elevated protein or RNA levels of IL-17 (and/or IL-13) and a second marker selected from: SAA, LCN2, and YKL-40, and treating the subject with an IL-17 (or IL-13) inhibitor or IL-17 (or IL-13) receptor inhibitor. In other embodiments, methods, compositions, and systems are provided for detecting a biomarker in a sample from a subject that has been treated with an IL-17 (or IL-13) inhibitor or IL-17 (or IL-13) receptor inhibitor, where the biomarker is SAA, LCN2, or YKL-40, and where at least one action is performed, such as identifying elevated levels of the biomarker in the biological sample and treating the subject with an IL-17 (or IL-13) inhibitor and/or IL-17 (or IL-13) receptor inhibitor.

In some embodiments, the kits are provided for the detection and characterization of IL-17 and/or IL-13 related conditions, such as asthma, autoimmune diseases, and cancer hypertension and/or responsiveness to MR antagonists. In some embodiments, the kits contain reagents for detecting biomarkers described herein (e.g., primers, probes, and/or antibodies specific for these biomarkers), in addition to other detection reagents, amplification reagents, stabilization reagents, purification reagents, buffers, controls, etc. In certain embodiments, kits contain all of the components necessary to perform a detection assay, including all controls, directions for performing assays, and any necessary software for analysis and presentation of results. In other embodiments, one or more reagents for performing the assay are not supplied in the kit and are instead supplied by the user. In some embodiments, kits comprise instructions (e.g. written, digital, and/or online) to perform assays for detecting and treating IL-17 and/or IL-13 related conditions, such as asthma, autoimmune diseases, and cancer.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, data is presented that will benefit the clinician, who is not likely to be trained in molecular biology, and need not understand the raw data. The data is presented directly to the clinician in a useful form. The clinician is then able to immediately utilize the information in order to optimize the care of the subject, such as providing or discontinuing IL-17 or IL-13 inhibitor (or receptor inhibitor) treatment.

Provided herein are any methods capable of receiving, processing, and transmitting the information to and from laboratories conducting the assays described herein, information providers, medical personal, and subjects. For example, in some embodiments, a sample (e.g., plasma sample) is obtained from a subject and submitted to a profiling service (e.g., clinical lab at a medical facility, independent testing facility, etc.), located in any part of the world (e.g., in a state or country different than where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a blood sample) and directly send it to a profiling center. Where the sample comprises previously determined biological information, the information may be sent directly to the profiling service by the subject (e.g., an information card containing the information may be scanned by a computer and the data transmitted to a computer of the profiling center using an electronic communication system). Once received by the profiling service, the sample is processed and a profile is produced, specific for the diagnostic or prognostic information desired for the subject.

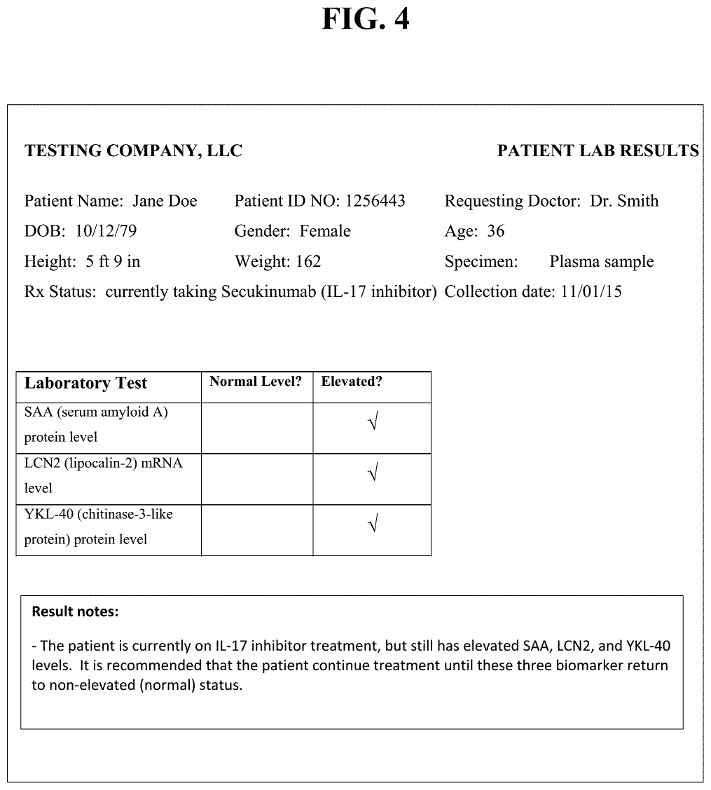

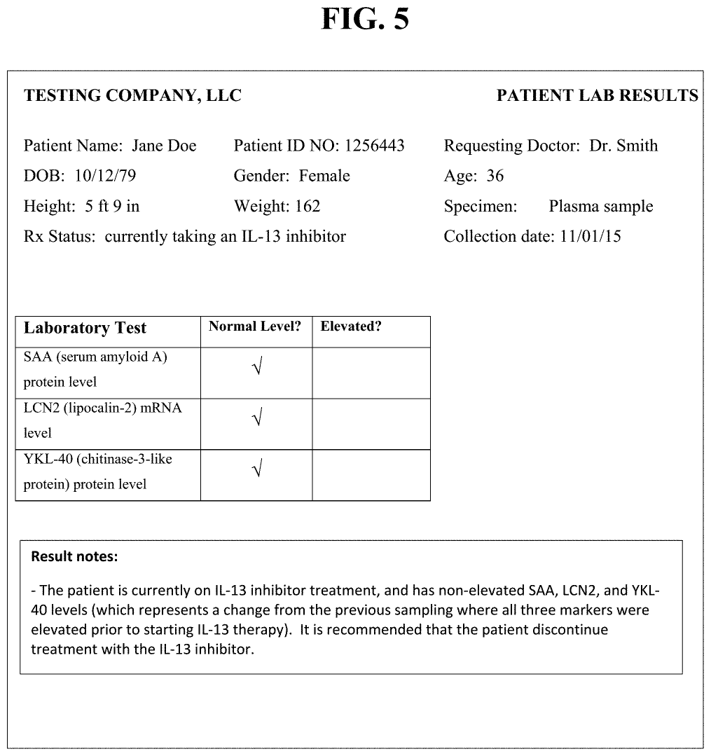

The profile data is then prepared in a format suitable for interpretation by a treating clinician. For example, rather than providing raw data, the prepared format may represent a diagnosis or risk assessment (e.g., likelihood of response to IL-17 and/or IL-13 inhibitor therapy) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. Examples of such reports are provided in FIGS. 4 and 5.

In some embodiments, the information from a biomarker assay is first analyzed at the point of care or at a regional facility. The raw data is then sent to a central processing facility for further analysis and/or to convert the raw data to information useful for a clinician or patient. The central processing facility provides the advantage of privacy (e.g., all data is stored in a central facility with uniform security protocols), speed, and uniformity of data analysis. The central processing facility can then control the fate of the data following treatment of the subject (e.g., after treatment with an IL-17 or IL-13 inhibitor or receptor inhibitor). For example, using an electronic communication system, the central facility can provide data to the clinician, the subject, or researchers. In some embodiments, the subject is able to directly access the data using the electronic communication system. The subject may chose further intervention or counseling based on the results. In some embodiments, the data is used for research use.

The present disclosure is not limited by the type of assay used to detect the biomarkers described herein (e.g., SAA, LCN2, YKL-40, IL-13, Csf3, Plat, Lgals3, Prl2c2, S100a8, and Gdf6, or a marker from Table 1 or combinations thereof). In certain embodiments, an immunoassay is employed for detecting one or more of the biomarkers. Any suitable assay known in the art can be used, including commercially available assays. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc.

In embodiments in which a biomarker is an RNA (e.g., mRNA), detection and/or quantification reagents may comprise primers (e.g., for amplification, reverse transcription, etc.) or probes (e.g., detectably-labeled (e.g., optically-labeled, fluorescently labeled, etc.) oligonucleotides) that bind (e.g., specifically) to the biomarker. In such embodiments, detection and/or quantification may be achieved by, for example, RT-PCR, qPCR, Northern blot analysis, an enzymatic cleavage assay (e.g., INVADER, Hologic, Inc.; See e.g., U.S. Pat. Nos. 5,846,717, 6,090,543; 6,001,567; 5,985,557; and 5,994,069; each of which is herein incorporated by reference), a hybridization assay (e.g., TaqMan assay (Life Technologies; See e.g., U.S. Pat. Nos. 5,962,233 and 5,538,848, each of which is herein incorporated by reference), etc.

In some embodiments, antibodies (e.g., monoclonal antibodies or antigen binding portions thereof) are used for the detection of an inflammatory marker protein (e.g., human: SAA, LCN2, YKL-40, IL-13, Csf3, Plat, Lgals3, Prl2c2, S100a8, and Gdf6). Such antibodies include, for example, the following monoclonal antibodies or antigen binding portions thereof: anti-SAA, anti-LCN2, anti-YKL-40, anti-IL-13, anti-Csf3, anti-Plat, anti-Lgals3, anti-Pr12c2, anti-S100a8, and anti-Gdf6, where such antibodies bind to the human version of the proteins. The antibodies may be prepared using various immunogens. In one embodiment, the immunogen used it generated antibodies is a an inflammatory marker protein or portion thereof selected from the following: human SAA (accession no. NP_000322; e.g., mature protein aa19 . . . 122); human LCN2 (accession no. NP_005555.2; e.g., aa21 . . . 198); human YKL-40 (accession no. NP_001267; e.g., aa22 . . . 383); human CSF3 (accession no. NP_000750; e.g., aa31 . . . 207); human PLAT (accession no. NP_000921; e.g., aa36 . . . 562); human LGALS3 (accession no. NP_002297; e.g., aa1 . . . 250); human S100a8 (accession no. NP_001306125; e.g., aa1 . . . 117); and human GDF6 (accession no. NP_001001557; e.g., aa 336 . . . 455). All of the foregoing accession numbers are herein incorporated by reference as if fully set forth herein. Such antibodies include, but are not limited to polyclonal, monoclonal, chimeric, single chain, Fab fragments, and Fab expression libraries.

Various procedures known in the art may be used for the production of polyclonal antibodies directed against an inflammatory marker as recited above and herein. For the production of antibody, various host animals can be immunized by injection with the peptide corresponding to an inflammatory marker epitope including but not limited to rabbits, mice, rats, sheep, goats, etc. In certain embodiments, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette Guerin) and Corynebacterium parvum).

For preparation of monoclonal antibodies directed toward an inflammatory marker protein, it is contemplated, in certain embodiments, that any technique that provides for the production of antibody molecules by continuous cell lines in culture will find use with the present disclosure (See e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Kohler and Milstein, Nature 256:495 497 [1975]), as well as the trioma technique, the human B cell hybridoma technique (See e.g., Kozbor et al., Immunol. Tod., 4:72 [1983]), and the EBV hybridoma technique to produce human monoclonal antibodies (Cole et al., in Monoclonal Antibodies and Cancer Therapy, Alan R. Liss, Inc., pp. 77 96 [1985]).

In certain embodiments, monoclonal antibodies are produced in germ free animals utilizing technology such as that described in PCT/US90/02545. Furthermore, it is contemplated that human antibodies may be generated by human hybridomas (Cote et al., Proc. Natl. Acad. Sci. USA 80:2026 2030 [1983]) or by transforming human B cells with EBV virus in vitro (Cole et al., in Monoclonal Antibodies and Cancer Therapy, Alan R. Liss, pp. 77 96 [1985]).

In addition, it is contemplated that techniques described for the production of single chain antibodies (U.S. Pat. No. 4,946,778; herein incorporated by reference) will find use in producing an inflammatory marker specific single chain antibodies. An additional embodiment of the invention utilizes the techniques described for the construction of Fab expression libraries (Huse et al., Science 246:1275 1281 [1989]) to allow rapid and easy identification of monoclonal Fab fragments with the desired specificity for An inflammatory marker.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. As is well known in the art, the immunogenic peptide should generally be provided free of the carrier molecule used in any immunization protocol. For example, if the peptide was conjugated to KLH, it may be conjugated to BSA, or used directly, in a screening assay to detect an inflammatory marker in a sample. The foregoing antibodies can be used to detect an inflammatory marker in a biological sample from an individual. The biological sample can be a biological fluid, such as, but not limited to, blood, serum, plasma, interstitial fluid, urine, cerebrospinal fluid, and the like, containing cells.

The biological samples can then be tested directly for the presence of an inflammatory marker herein using an appropriate strategy (e.g., ELISA or radioimmunoassay) and format (e.g., microwells, dipstick (e.g., as described in International Patent Publication WO 93/03367), etc. Alternatively, proteins in the sample can be size separated (e.g., by polyacrylamide gel electrophoresis (PAGE), in the presence or not of sodium dodecyl sulfate (SDS), and the presence of an inflammatory marker detected by immunoblotting (Western blotting). Immunoblotting techniques are generally more effective with antibodies generated against a peptide corresponding to an epitope of a protein, and hence, are particularly suited to the methods and compositions disclosed herein.

In certain embodiments, a commercial kit is employed to detect one or more of the inflammatory markers described herein. For example, the following commercial ELISA kits may be employed (e.g., for human plasma analysis): 1) Lcn2 Human ELISA kit (BIOTANG; Cat. No. Hu9225); 2) SAA3 Human ELISA kit (ABCAM, Cat. No. ab100635); and 3) YKL40 Human ELISA kit (MBL Cat. No. CY-8088).

In some embodiments, an inflammatory marker is detected with an immunoassay such as: 1) a sandwich immunoassay (e.g., monoclonal, polyclonal and/or DVD-Ig sandwich immunoassays or any variation thereof (e.g., monoclonal/DVD-Ig or DVD-Ig/polyclonal), including chemiluminescence detection, radioisotope detection (e.g., radioimmunoassay (RIA)) and enzyme detection (e.g., enzyme immunoassay (EIA) or enzyme-linked immunosorbent assay (ELISA) (e.g., Quantikine ELISA assays, R&D Systems, Minneapolis, Minn.))), 2) a competitive inhibition immunoassay (e.g., forward and reverse), 3) a fluorescence polarization immunoassay (FPIA), 4) an enzyme multiplied immunoassay technique (EMIT), 5) a bioluminescence resonance energy transfer (BRET), 6) a homogeneous chemiluminescent assay, 7) a SELDI-based immunoassay, 8) chemiluminescent microparticle immunoassay (CMIA) and 9) a clinical chemistry colorimetric assay (e.g., IMA, creatinine for eGFR determination and LC-MS/MS). (See, e.g., Tietz Textbook of Clinical Chemistry and Molecular Diagnostics. 4th Edition, edited by C A Burtis, E R Ashwood and D E Bruns, Elsevier Saunders, St. Louis, Mo., 2006.).

Further, if an immunoassay is being utilized, any suitable detectable label as is known in the art can be used. For example, the detectable label can be a radioactive label (such as 3H, 125I, 35S, 14C, 32P, and 33P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous or heterogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

The chemiluminescent signal that is generated can be detected using routine techniques known to those skilled in the art. Based on the intensity of the signal generated, the amount of analyte in the sample can be quantified. Specifically, the amount of analyte in the sample is proportional to the intensity of the signal generated. The amount of analyte present can be quantified by comparing the amount of light generated to a standard curve for the analyte or by comparison to a reference standard. The standard curve can be generated using serial dilutions or solutions of known concentrations of analyte by mass spectroscopy, gravimetric methods, and other techniques known in the art.

Immunoassays can generally be conducted using any format known in the art, such as, but not limited to, a sandwich format. Specifically, in one immunoassay format, at least two antibodies are employed to separate and quantify an inflammatory marker or a fragment thereof in a sample. More specifically, the at least two antibodies bind to different epitopes on an inflammatory marker (or a fragment thereof) forming an immune complex, which is referred to as a “sandwich.” Generally, in the immunoassays, one or more antibodies can be used to capture the an inflammatory marker (or a fragment thereof) in the test sample (i.e., these antibodies are frequently referred to as a “capture” antibody or “capture” antibodies) and one or more antibodies can be used to bind a detectable (namely, quantifiable) label to the sandwich (i.e., these antibodies are frequently referred to as the “detection antibody,” the “detection antibodies,” the “conjugate,” or the “conjugates”). Thus, in the context of a sandwich immunoassay format, an antibody (or a fragment, a variant, or a fragment of a variant thereof) can be used as a capture antibody, a detection antibody, or both. For example, one DVD-Ig having a domain that can bind a first epitope on an inflammatory marker (or a fragment thereof) can be used as a capture antibody and/or another DVD-Ig having a domain that can bind a second epitope on an inflammatory marker (or a fragment thereof) can be used as a detection antibody. In this regard, a DVD-Ig having a first domain that can bind a first epitope on an analyte (or a fragment thereof) and a second domain that can bind a second epitope on an analyte (or a fragment thereof) can be used as a capture antibody and/or a detection antibody. Alternatively, one DVD-Ig having a first domain that can bind an epitope on a first analyte (or a fragment thereof) and a second domain that can bind an epitope on a second analyte (or a fragment thereof) can be used as a capture antibody and/or a detection antibody to detect, and optionally quantify, two or more analytes.

Generally speaking, in an immunoassay, a sample being tested for (for example, suspected of containing) an inflammatory marker (or a fragment thereof) can be contacted with at least one capture antibody (or antibodies) and at least one detection antibody (which can be a second detection antibody or a third detection antibody or even a successively numbered antibody, e.g., as where the capture and/or detection antibody comprise multiple antibodies) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody.

In the sandwich assay format, described above, a sample suspected of containing an inflammatory marker (or a fragment thereof) is first brought into contact with at least one first capture antibody under conditions that allow the formation of a first antibody/an inflammatory marker complex. If more than one capture antibody is used, a first capture antibody/an inflammatory marker complex comprising two or more capture antibodies is formed. In a sandwich assay, the at least one capture antibody, are generally used in molar excess amounts of the maximum amount of an inflammatory marker (or a fragment thereof) expected in the test sample. For example, from about 5 μg to about 1 mg of antibody per mL of buffer (e.g., microparticle coating buffer) can be used.

In contrast, competitive inhibition immunoassays, which are often used to measure small analytes because binding by only one antibody is required, comprise sequential and classic formats. In a sequential competitive inhibition immunoassay, a capture antibody to an analyte of interest (e.g., an inflammatory marker protein) is coated onto a well of a microtiter plate or other solid support. When the sample containing the analyte of interest is added to the well, the analyte of interest binds to the capture antibody. After washing, a known amount of labeled analyte (e.g., acridinium, biotin or horseradish peroxidase (HRP)) is added to the well. A substrate for an enzymatic label is necessary to generate a signal. An example of a suitable substrate for HRP is 3,3′, 5,5′-tetramethylbenzidine (TMB). After washing, the signal generated by the labeled analyte is measured and is inversely proportional to the amount of analyte in the sample. In a classic competitive inhibition immunoassay, an antibody to an analyte of interest is coated onto a solid support (e.g., a well of a microtiter plate). However, unlike the sequential competitive inhibition immunoassay, the sample and the labeled analyte are added to the well at the same time. Any analyte in the sample competes with labeled analyte for binding to the capture antibody. After washing, the signal generated by the labeled analyte is measured and is inversely proportional to the amount of analyte in the sample.

In general, the concentration of an inflammatory marker or a fragment thereof in the test sample is determined by appropriate means, such as by use of a standard curve that has been generated using serial dilutions of analyte or a fragment thereof of known concentration. Other than using serial dilutions of analyte or a fragment thereof, the standard curve can be generated gravimetrically, by mass spectroscopy and by other techniques known in the art.

In some embodiments, in a chemiluminescent microparticle assay employing the ARCHITECT analyzer, the conjugate diluent pH may be about 6.0+/−0.2, the microparticle coating buffer may be maintained at about room temperature (i.e., at from about 17 to about 27.degree. C.), the microparticle coating buffer pH may be about 6.5+/−0.2, and the microparticle diluent pH may be about 7.8+/−0.2. Solids preferably are less than about 0.2%, such as less than about 0.15%, less than about 0.14%, less than about 0.13%, less than about 0.12%, or less than about 0.11%, such as about 0.10%. Of course, these ranges or numbers may be altered in order to enhance such properties of the assay including, for example, reduction in background interference, increased sensitivity, increased specificity, etc.

In some embodiments, Fluorescence polarization immunoassays (FPIAs) are used to detect one or more of the inflammation markers herein. FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (i.e., a compound that is not bound to an antibody) is excited by linearly polarized light, its rotation is much faster than the corresponding tracer-antibody conjugate produced in a competitive binding immunoassay. FPIAs are advantageous over RIAs inasmuch as there are no radioactive substances requiring special handling and disposal. In addition, FPIAs are homogeneous assays that can be easily and rapidly performed.

In certain embodiments, provided herein are inflammatory marker diagnostic kits comprising the immunodiagnostic reagents described herein and another component, such as reagents for detecting a second (or third) inflammatory marker or instructions for the use of the immunodiagnostic reagents in immunoassays for determining the presence of an inflammatory marker in a test sample by detecting the presence of two or more an inflammatory marker proteins and/or anti-an inflammatory marker antibodies in such a sample. For example, the kit can comprise instructions for assaying the test sample for anti-an inflammatory marker antibody by immunoassay. While certain embodiments employ chemiluminescent microparticle immunoassay for assaying the test sample, it should be understood that the antigens and antibodies used in the immunoassays of the present invention may be used in any other immunoassay formats known to those of skill in the art for determining the presence of an inflammatory marker in a test sample. The instructions can be in paper form or computer-readable form, such as a disk, CD, DVD, or the like. Alternatively or additionally, the kit can comprise a calibrator or control, e.g., purified, and optionally lyophilized, anti-an inflammatory marker antibody or antigen, and/or at least one container (e.g., tube, microtiter plates or strips, which can be already coated with one or more of the capture components (antigens and/or antibodies) of the immunoassay) for conducting the assay, and/or a buffer, such as an assay buffer or awash buffer, either one of which can be provided as a concentrated solution, a substrate solution for the detectable label (e.g., an enzymatic label), or a stop solution. In certain embodiments, the kit comprises all components, i.e., reagents, standards, buffers, diluents, etc., which are necessary to perform the assay. In specific embodiments, the components are individually presented in the kit such that the immunoassay may be performed as a capture-on-the-fly type combination immunoassay in which the solid support is coated with an agent that allows binding of the capturing moiety (e.g., a-biotinylated antigen or a biotinylated antibody) and the kit further comprises each of the individual capture and detection antigen pairs and the biotinylated capture antibodies in one container and a second container provides the detection antibody conjugate. The instructions for conducting the assay also can include instructions for generating a standard curve or a reference standard for purposes of quantifying anti-inflammatory marker antibody.

Any antibodies, which are provided in the kit, such as anti-IgG antibodies and anti-IgM antibodies, can also incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates. In certain immunoassays, there are two containers provided. In the first container is provided at least a first, second and third pair of antigens, wherein the first antigen in each pair is a capture antigen from a given an inflammatory marker protein that is biotinylated and the second antigen in each pair is a detection antigen from the same protein as the first antigen but is labeled with a detectable label (e.g., it is acridinylated) as well as one or more biotinylated antibodies designed for detecting one or more an inflammatory marker antigens from a test sample; and in the second container is provided the antibody that forms the conjugation partner for detection of the antigen that is captured by the biotinylated antibodies from the first container. It is contemplated that where there are multiple biotinylated antibodies in the first container, the multiple antibodies that form the conjugation partners may be present in a single container or individual containers for each different antigen detecting conjugate antibody.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

### EXPERIMENTAL

### Example 1

### Detection of IL-17 and IL-13 Related Biomarkers is Asthma

Patients with TH2-high asthma have greater improvement in lung function in response to inhaled corticosteroids compared with patients with “TH2-low” asthma. The underlying mechanisms of the TH2-low phenotype remain unknown, but these patients usually display predominant bronchial neutrophilic inflammation. In human tissues, IL-17A levels correlate with increasing asthma severity and in some studies were associated with neutrophilic inflammation. Consistent with the literature, it was found that IL-17A levels were elevated in the serum of asthma patients, trending higher with severity. It is intriguing that populations of CD4+ T cells that express IL-17A and TH2 cytokines have also been found in the lungs of mice with allergic airway disease and blood from asthmatic patients. While IL-17A can directly impact multiple aspects of asthma pathogenesis (including inflammatory response of epithelial cells, smooth muscle cell hyperplasia and smooth muscle contraction), it was also observed that there was strong synergy between IL-17A and IL-13 on smooth muscle cells for gene induction and cell proliferation. While the present invention is not limited to any particular mechanism, and an understanding of the mechanism is not necessary to practice the invention, based on these findings, it was hypothesized that IL-17A either acts alone or in synergy with IL-13 to impact on airway epithelial cells and smooth muscle cells to induce inflammation, smooth muscle cell hyperplasia and smooth muscle contraction. A pre-specified subset analysis suggested that patients with “high-reversibility” asthma might benefit from anti-IL-17 therapy. Measures of TH17 signatures combined with measures of the effects of IL-17A and IL-17A+IL-13 on target cells are valuable for selecting subjects with appropriate IL-17A-related endotypes for inclusion in clinical trials of IL-17 inhibitors (e.g., small molecule inhibitors). Therefore, one goal was to identify specific biomarkers to endotype severe asthmatics with TH17-IL-17A or TH2/TH17-IL-17A signatures for IL-17A-targeted therapy.

### Results

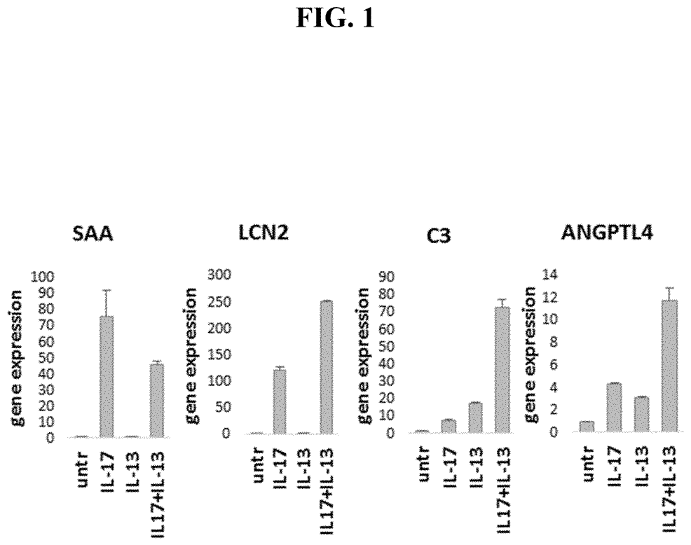

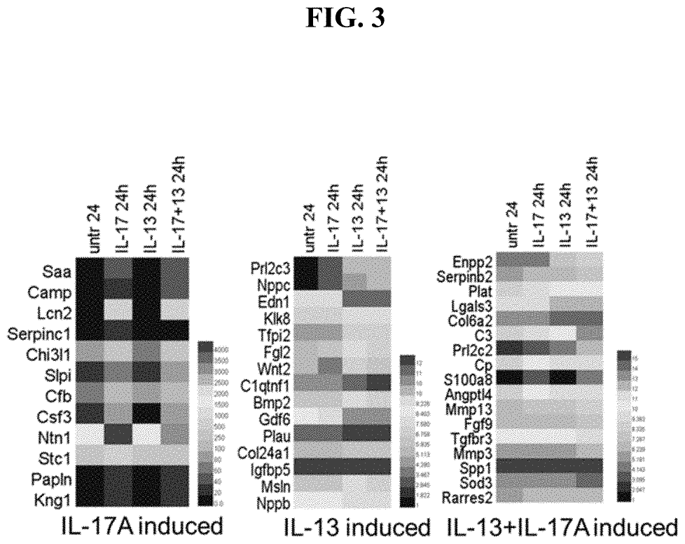

Airway smooth muscle cells are highly responsive to IL-17: Increased ASM mass is a feature of airway remodeling in severe asthma. RNA sequencing (RNA-seq) identified several genes highly induced by IL-17A in ASM cells, including Serum Amyloid A (SAA), LCN2 and YKL-40 (Chi3l1) (FIG. 1 and FIG. 3). While SAA, LCN2 and YKL-40 (Chi3l1) may serve as biomarkers of TH17/IL-17A-driven asthma, SAA has been shown to promote TH17 cell polarization, possibly providing a positive feedback on TH17/IL-17A axis. IL-17A and IL-13 showed strong synergy in ASM cells for induction of certain inflammatory genes (including LCN2, C3 and Angptl4) (FIG. 1).

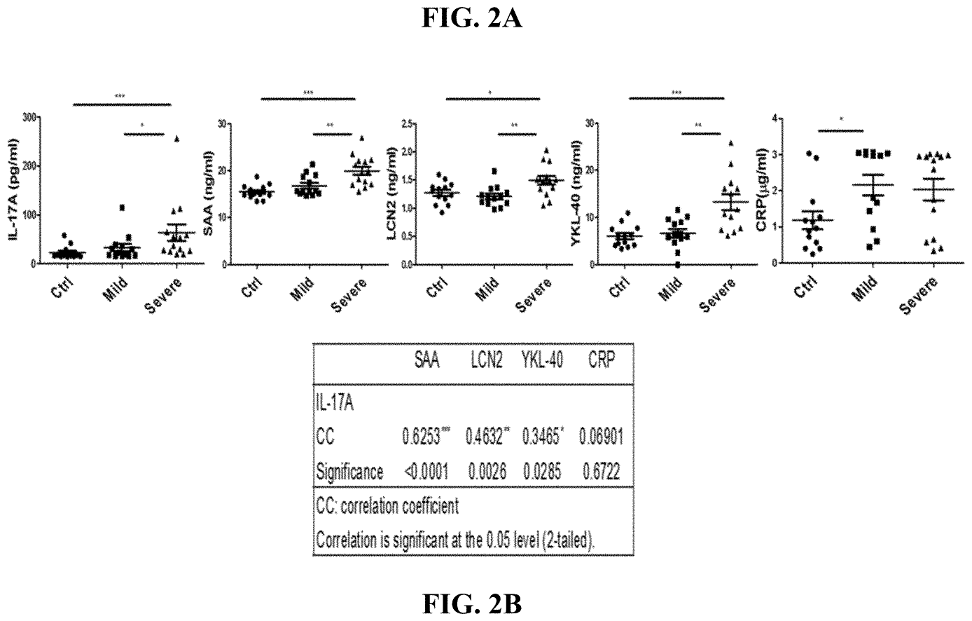

IL-17A, SAA, LCN2 and YKL-40 (Chi3l1) are elevated in severe asthmatics: Consistent with the literature, we found that IL-17A levels were significantly elevated in the serum of asthmatics compared to that of healthy controls, trending even higher in severe asthmatics (FIG. 2A). To identify potential biomarkers for endotyping potential IL-17A responders, the RNA-seq data of ASM cells was analyzed (FIG. 3), with a focus on the IL-17A-induced genes that produce secreted proteins, since these proteins have the potential to be detected in serum. As the genes for the secreted proteins SAA, LCN2 and YKL-40 (Chi3l1) were highly induced by IL-17A (FIG. 2A and FIG. 3), their levels were measured in serum collected from healthy controls and mild and severe asthmatics. SAA, LCN2 and YKL-40 (Chi3l1) were elevated in serum of severe asthmatics compared to that of mild asthmatics or healthy controls, correlating with serum IL-17A levels (FIG. 2B). Although CRP was higher in asthmatics, CRP was not induced by IL-17A, and CRP levels did not correlate with serum IL-17A, excluding CRP as a potential biomarker for endotyping potential IL-17A responders (FIGS. 2A and B).

Identification of IL-17A and IL-17A+IL-13 target genes in human ASM cells: By performing RNA-seq, several genes were identified that were highly induced by IL-17A in mouse ASM cells that were also elevated in the serum of patients with severe asthma, including Serum Amyloid A (SAA) and LCN2 (FIG. 2A and FIG. 3). The full list of genes from mouse ASM cells in FIG. 3 is presented in Table 1 below.

TABLE 1

| | | |
|---|---|---|
| Saa | serum amyloid A1 | IL-17A induced |
| Camp | cathelicidin antimicrobial peptide | IL-17A induced |
| Lcn2 | lipocalin-2 | IL-17A induced |
| Serpinc 1 | serpin peptidase inhibitor, clade C | IL-17A induced |
| | (antithrombin), member 1 | |
| Chi3l1 | chitinase 3 like 1 | IL-17A induced |
| Slpi | secretory leukocyte peptidase | IL-17A induced |
| | inhibitor | |
| Cfb | complement factor B | IL-17A induced |
| Csf3 | colony stimulating factor 3 | IL-17A induced |
| Ntn1 | netrin 1 | IL-17A induced |
| Stc1 | stanniocalcin 1 | IL-17A induced |
| Papln | papilin, proteoglycan-like sulfated | IL-17A induced |
| | glycoprotein | |
| Kng1 | kininogen 1 | IL-17A induced |
| Prl2c3 | prolactin family 2, subfamily c, | IL-13 induced |
| | member 3 | |
| Nppc | natriuretic peptide C | IL-13 induced |
| Edn1 | endothelin 1 | IL-13 induced |
| Klk8 | kallikrein related peptidase 8 | IL-13 induced |
| Tfpi2 | tissue factor pathway inhibitor 2 | IL-13 induced |
| Fgl2 | fibrinogen-like 2 | IL-13 induced |
| Wnt2 | wingless-type MMTV integration site | IL-13 induced |
| | family member 2 | |
| C1qtnf1 | C1q and tumor necrosis factor | IL-13 induced |
| | related protein 1 | |
| Bmp2 | bone morphogenetic protein 2 | IL-13 induced |
| Gdf6 | growth differentiation factor 6 | IL-13 induced |
| Plau | plasminogen activator, urokinase | IL-13 induced |
| Col24a1 | collagen, type XXIV, alpha 1 | IL-13 induced |
| Igfbp5 | insulin like growth factor binding | IL-13 induced |
| | protein 5 | |
| Msln | mesothelin | IL-13 induced |
| Nppb | natriuretic peptide B | IL-13 induced |
| Enpp2 | ectonucleotide | IL-13 + IL-17A |
| | pyrophosphatase/phosphodiesterase | induced |
| | 2 | |
| Serpinb2 | serpin peptidase inhibitor, clade B | IL-13 + IL-17A |
| | (ovalbumin), member 2 | induced |
| Plat | plasminogen activator, tissue | IL-13 + IL-17A |
| | | induced |
| Lgals3 | lectin, galactoside-binding, soluble, 3 | IL-13 + IL-17A |
| | | induced |
| Col6a2 | collagen, type VI, alpha 2 | IL-13 + IL-17A |
| | | induced |
| C3 | complement component 3 | IL-13 + IL-17A |
| | | induced |
| Prl2c2 | prolactin family 2, subfamily c, | IL-13 + IL-17A |
| | member 2 | induced |
| Cp | ceruloplasmin (ferroxidase) | IL-13 + IL-17A |
| | | induced |
| S100a8 | S100 calcium binding protein A8 | IL-13 + IL-17A |
| | | induced |
| Angptl4 | angiopoietin like 4 | IL-13 + IL-17A |
| | | induced |
| Mmp13 | matrix metallopeptidase 13 | IL-13 + IL-17A |
| | | induced |
| Fgf9 | fibroblast growth factor 9 | IL-13 + IL-17A |
| | | induced |
| Tgfbr3 | transforming growth factor beta | IL-13 + IL-17A |
| | receptor III | induced |
| Mmp3 | matrix metallopeptidase 3 | IL-13 + IL-17A |
| | | induced |
| Spp1 | secreted phosphoprotein 1 | IL-13 + IL-17A |
| | | induced |
| Sod3 | CXXC finger protein 1 | IL-13 + IL-17A |
| | | induced |
| Rarres2 | retinoic acid receptor responder | IL-13 + IL-17A |
| | (tazarotene induced) 2 | induced |

The RNA-seq data was further analyzed with a focus on the IL-17A-, IL-13- and IL-17A+IL-13-induced genes that produce secreted proteins (listed in the heatmap of FIG. 3), since these secreted proteins have the potential to be detected in the serum as biomarkers. The utility of the genes in Table 1 as biomarkers herein can be evaluated, for example, by measuring the level of these proteins in the plasma of asthmatic patients (mild and severe) and healthy controls.

### Material and Methods

### RNA Isolation and Real-Time PCR

Total RNA was isolated using TRIzol reagent (Invitrogen), according to the manufacturer's protocol A quantity amounting to 1 μg of total RNA was then used for the reverse transcription reaction using Super Script II-reverse transcriptase (Invitrogen). Quantitative real-time PCR was performed in AB 7300 RealTime PCR System, and the gene expression was examined by SYBR Green PCR Master Mix (Applied Biosystems). PCR amplification was performed in duplicates. The reaction protocol included preincubation at 95° C. to activate FastStart DNA polymerase for 10 min, amplification of 40 cycles that was set for 15 s at 95° C., and the annealing for 60 s at 60° C. The results were normalized with the housekeeping gene.

### RNA Sequencing

IL-17A, IL-13 and IL-17A+IL-13-induced transcripts were collected 0, 3 and 24 hours after stimulation from primary mouse ASM cells. Total RNA was extracted by the RNeasy method (Qiagen), and total RNA was qualified using an Agilent 2100 Bioanalyzer and prepared for sequencing using the Illumina RNA-sequencing kit as per the manufacturer's instructions. High-throughput sequencing was done on an Illumina HiSeq 2000 platform. Library construction and sequencing was performed by BGI (China).

Plasma Isolation from Human Blood and ELISA

Plasma was isolated using SepMate columns form the blood from healthy and asthmatic patients according to the manufacturer's protocol. The ELISAs for IL-17 (R&D), SAA (Abcam), LCN2 (Biotang), YKL40 (MBL), and CRP (Life Tech) were performed according to the manufacturer's protocols.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

1. A method comprising:
a) selecting a subject with symptoms of an inflammatory condition, that also has elevated levels of both IL-17 and a marker selected from the group consisting of human SAA, LCN2, YKL-40, IL-13, Csf3, Plat, Lgals3, Prl2c2, S100a8, and Gdf6; and
b) treating said subject with an IL-17 inhibitor, and/or an IL-17 receptor inhibitor,

2. The method of claim 1, wherein said inflammatory condition is asthma.

3. The method of claim 2, wherein said asthma is severe asthma.

4. The method of claim 1, wherein said marker is human SAA, human LCN2, or human YKL-40.

5. A method comprising:
a) receiving information that a subject has elevated levels of both IL-17 and a marker selected from the group consisting of human SAA, LCN2, YKL-40, IL-13, Csf3, Plat, Lgals3, Prl2c2, S100a8, and Gdf6; and
b) treating said subject with an IL-17 inhibitor, and/or an IL-17 receptor inhibitor.

6. The method of claim 5, wherein said subject has symptoms of an inflammatory condition.

7. The method of claim 6, wherein said inflammatory condition is asthma.

8. The method of claim 7, wherein said asthma is severe asthma.

9. The method of claim 5, wherein said marker is human SAA, human LCN2, or human YKL-40.

10-14. (canceled)

15. A method comprising:
a) receiving information that a subject has: i) has been treated with an exogenous IL-17 inhibitor and/or exogenous IL-17 receptor inhibitor, and ii) has an elevated level of a biomarker selected from the group consisting of: human SAA, LCN2, YKL-40, IL-13, Csf3, Plat, Lgals3, Prl2c2, S100a8, and Gdf6; and
b) treating said subject with an IL-17 inhibitor, and/or an IL-17 receptor inhibitor.

16. The method of claim 15, wherein said subject has symptoms of an inflammatory condition.

17. The method of claim 16, wherein said inflammatory condition is asthma.

18. The method of claim 17, wherein said asthma is severe asthma.

19. The method of claim 15, wherein said marker is human SAA, human LCN2, or human YKL-40.

20-23. (canceled)
